(19) United States
(12) Patent Application Publication
(10) Pub. No.: US 2012/0123230 A1
(43) Pub. Date: May 17, 2012

(54) ANALYTE MONITORING SYSTEMS AND METHODS OF USE

(75) Inventors: David BROWN, Carlsbad, CA (US); Paul M. DiPerna, Cardiff, CA (US)

(73) Assignee: TANDEM DIEBETES CARE, INC., San Diego, CA (US)

(21) Appl. No.: 13/272,111

(22) Filed: Oct. 12, 2011

Related U.S. Application Data
(60) Provisional application No. 61/392,858, filed on Oct. 13, 2010.

(51) Int. Cl. A61B 5/1455; A61M 5/168; A61B 5/145

(52) U.S. Cl. 600/316; 600/309; 600/365; 600/347; 604/66

(57) ABSTRACT

Analyte monitoring systems and methods that make interstitial fluid from a patient's body available to one or more sensors disposed outside the patient's body. The monitoring systems and methods may be used in conjunction with medicament dispensing systems and methods in order to provide a feedback loop for continuous sensing of analyte levels and corresponding dispensing of medicament based on sensed analyte levels. Dispensing or pumping systems or portions thereof may be used to move a patient's interstitial fluid into communication with the one or more sensors.

### RELATED APPLICATIONS

This application claims priority under 35 U.S.C. section 119(e) from U.S. Provisional Patent Application Ser. No. 61/392,858, filed Oct. 13, 2010, by D. Brown et al., titled Analyte Monitoring Systems and Methods of Use, which is incorporated by reference herein in its entirety. This application also hereby incorporates by reference in their entirety each of the following commonly owned patents and patent applications: U.S. patent application Ser. No. 12/846,688, entitled “Infusion Pump System with Disposable Cartridge Having Pressure Venting and Pressure Feedback”, filed Jul. 29, 2010 by P. DiPerna et al.; U.S. patent application Ser. No. 12/846,720, entitled “Infusion Pump System with Disposable Cartridge Having Pressure Venting and Pressure Feedback”, filed Jul. 29, 2010 by P. DiPerna et al.; U.S. patent application Ser. No. 12/846,734, entitled “Infusion Pump System with Disposable Cartridge Having Pressure Venting and Pressure Feedback”, filed Jul. 29, 2010 by E. Verhoef et al.; U.S. patent application Ser. No. 12/846,706, entitled “Infusion Pump System with Disposable Cartridge Having Pressure Venting and Pressure Feedback”, filed Jul. 29, 2010, by M. Michaud et al.; U.S. patent application Ser. No. 12/846,733, entitled “Infusion Pump System with Disposable Cartridge Having Pressure Venting and Pressure Feedback”, filed Jul. 29, 2010, by M. Michaud et al.; PCT Patent Application No. PCT/US2010/043789 entitled “Infusion Pump System with Disposable Cartridge Having Pressure Venting and Pressure Feedback”, filed Jul. 29, 2010, by P. DiPerna et al; U.S. patent application Ser. No. 12/714,299, entitled “Methods and Devices for Determination of Flow Reservoir Volume”, filed Feb. 26, 2010 by M. Rosinko et al.; U.S. Pat. No. 7,008,403, entitled “Infusion Pump and Method for Use” by S. Mallett; U.S. Pat. No. 7,341,581, entitled “Infusion Pump and Method for Use” by S. Mallet; U.S. Pat. No. 7,374,556, entitled “Infusion Pump and Method for Use”, by S. Mallet; U.S. Patent Application Publication No. 2007/0264130, entitled “Infusion Pumps and Method for Use”, filed May 4, 2007 by S. Mallett; and U.S. Patent Application Publication No. 2009/0191067, entitled “Two Chamber Pumps and Related Methods”, filed Jan. 25, 2008 by P. DiPerna.

### FIELD OF THE INVENTION

This disclosure is directed to systems, devices and methods for monitoring bodily analytes such as glucose or other substances. In some cases, the systems, devices and methods may be used for infusing a material such as a medicament; e.g., insulin, into a body in need thereof. The systems, devices and methods disclosed herein are not limited to monitoring glucose and delivering insulin but may be directed to monitoring any number of analytes and delivering any number of molecules and types of molecules to the body.

### BACKGROUND

There are many applications in academic, industrial, and medical fields, as well as others, that may benefit from devices and methods that are capable of accurately and controllably delivering fluids, including liquids and gases that have a beneficial effect when administered in known and controlled quantities. This may be particularly true in the medical field where much of the treatment for a large percentage of patients includes the administration of a known amount of a substance at predetermined intervals. The treatment of diabetes often involves just such a regimented dosage of materials, in particular, the administration of insulin. In addition, the administration of insulin for a diabetic patient is one of a few medical indications wherein the patient routinely administers the medicament to themselves by a subcutaneous modality, such as a hypodermic syringe injection. As such, providing a patient with the means to safely, reliably and comfortably administer required doses of medication may be particularly important in order to facilitate patient compliance and accurate treatment of the condition.

Blood glucose is an important factor for metabolism and the provision of energy and proper organ functioning in mammals. The accurate regulation of blood glucose is, therefore, an essential task necessary for the well being of the mammal. For instance, the neurons of the brain of an organism depend on glucose for fueling their functioning. Hence, blood glucose levels are typically regulated by feedback loops between the brain and the pancreas. The pancreas functions in response to various hormones released by the brain by itself releasing hormones that regulate the uptake, e.g., storage, of blood sugar, or the release of stored blood sugar. For instance, two essential hormones in the regulation of blood sugar levels are insulin and glucagon, both of which are synthesized by specialized cells in the pancreas. Specifically, the β cells of the islets of Langerhans function to synthesize insulin, while the α cells of the islets of Langerhans function to synthesize glucagon.

Maintaining appropriate blood glucose homeostasis is an important factor for promoting the length and quality of life. However, there are many factors that affect the body's ability to maintain such homeostasis. For instance, factors such as the body's ability to produce or respond to insulin, one's physiological condition and/or health, the quantity and type of food one eats, one's metabolic rate, activity level, the types of activities and the exertion level in which one engages, as well as other such factors that make up a person's daily life and/or routine, all play important roles in effecting the body's ability to maintain homeostasis.

Continuous subcutaneous insulin injection and/or infusion therapy may be initiated for the replacement of insulin and thereby the treatment of diabetes. Such therapy may include the regular and/or continuous injection or infusion of insulin into the skin of a person suffering from diabetes. Injection is the traditional and most common method for administering insulin. Typically the diabetic will measure his or her blood glucose level, and depending on the level thereof may prepare a syringe or injection pen with insulin to be injected transdermally into the body. However, recently, insulin injecting pumps have been developed for the administration of insulin for those suffering from both type I and II diabetes. Insulin pumps are medical devices used for the administration of insulin in the treatment of diabetes and offer an alternative to multiple daily injections of insulin by an insulin syringe or an insulin pen. They also allow for continuous insulin therapy by having the ability to deliver low level continuous basal rates to the patient and larger bolus and correction bolus doses as required per the above conditions.

In addition to delivering materials such as insulin, there are various methods and devices for measuring the concentration of an analyte such as glucose in the body of a diabetic patient. For instance, finger-prick capillary samples of blood are often used to measure the concentration of glucose in the patient's blood to determine whether insulin therapy is needed, and if so, how much insulin to infuse into the patient's body. Blood samples obtained by this method typically are applied to a reagent strip for analysis in a meter such as, e.g., those systems sold by Agamatrix, Inc. of Salem, N.H. Other methods and devices for continuously or semi-continuously monitoring analytes such as glucose in, e.g., the subcutaneous interstitial fluid (ISF) include those that incorporate sensors such as glucose oxidate (GOx)-based electrodes. Some such devices may include the Guardian® Real-Time Continuous Glucose Monitoring System sold by Medtronic, Inc. of St. Paul, Minn. and the FreeStyle Navigator® system sold by Abbott Laboratories, Abbott Park, Ill. Some such devices and methods may also be described in, e.g., U.S. Pat. No. 6,360,888 to Mclvor et al., U.S. Pat. No. 6,892,085 to Mclvor et al., and U.S. Pat. No. 6,881,551 to Heller et al., each of which is incorporated herein by reference in its entirety. These sensors consist of a subcutaneously implantable, needle-type amperometric enzyme electrode. Other continuous or semi-continuous monitoring techniques include the use of reverse iontophoresis-based sensors as detailed in, e.g., U.S. Pat. No. 6,391,643 to Chen et al., the entirety of which is incorporated herein by reference, and microdialysis-based technologies as described in, e.g., U.S. Pat. No. 6,091,976, the entirety of which is incorporated herein by reference.

Continuous or semi-continuous glucose monitoring systems may have the advantage of providing a patient and caregiver with accurate and timely information regarding the patient's glucose level compared with the use of test strips so to allow for the more accurate and safe delivery of insulin to the patient when needed. For instance, continuous glucose monitoring systems may prevent the patient and caregiver from missing glucose levels that may be significantly higher or lower than optimal as may occur in between tests obtained using test strips. Such systems may be used in connection with infusion pumps to deliver care to diabetes patients in a “closed loop” or “semi-closed loop” fashion in which a communications link connects the monitor and infusion pump to deliver optimal care to the patient via, e.g., a controller. Such systems are described in, e.g., U.S. Pat. No. 6,558,351, the entirety of which is incorporated herein by reference. Other such systems and methods are described in U.S. Patent Application Serial No. US 2010/0256593, published Oct. 7, 2010 to Yodfat et al., entitled “Analyte Monitoring and Fluid Dispensing System”, the entirety of which is hereby incorporated by reference. What have been needed are systems and methods for safely monitoring analytes in a patient's body over a period of time which are convenient and reliable.

### SUMMARY

System, device and method embodiments for continuous or semi-continuous monitoring of levels of concentration of one or more analytes such as, e.g., glucose, within a patient's body are disclosed herein. In some embodiments, systems, devices and methods incorporate one or more continuous or semi-continuous sensors such as a glucose sensor placed within a fluid path of a cannula placed through the skin in the subcutaneous space, in the intramuscular space, in the dermal layers, or, with a longer cannula perhaps, a venous blood vessel. The sensor or sensors in some embodiments, however, may not be in direct contact with the body, and therefore may not be subjected to the challenges associated with sensors based in the body. The sensor itself may be of any well-known device types, including but not limited to; glucose oxidase (GOx) based electrodes, fluorescence based devices, or other devices as described variously herein and/or as known in the art. In some embodiments, the sensor can be placed near the cannula.

In some embodiments, measurements taken by a sensor and other information that may be processed by the system may be transmitted to, e.g., a recording medium and/or a user interface to present the measurements and/or other information to a patient or caregiver. Such information may also or in addition be transmitted by a processor via a communications link (e.g., wireless, optical, wired, etc.) to a remote device such as a device for presenting the measurements and/or other information to a patient or caregiver, or a remote infusion device such as an insulin pump, such as those described variously in PCT Patent Application No. PCT/US2010/043789 entitled “Infusion Pump System with Disposable Cartridge Having Pressure Venting and Pressure Feedback”, filed Jul. 29, 2010, by P. DiPerna et al.

In some embodiments, the measurements taken by the sensor or sensors and other information that may be processed by the system can be transmitted to, e.g., a recording medium and/or a user interface to present the measurement to a patient or caregiver. Such information can also or in addition be transmitted by a processor via a communications link (e.g., wireless, optical, wired, etc.) to an integrated infusion device such as an insulin pump, such as those described variously in PCT Patent Application No. PCT/US2010/043789 entitled “Infusion Pump System with Disposable Cartridge Having Pressure Venting and Pressure Feedback”, filed Jul. 29, 2010, by P. DiPerna et al.

Some embodiments of the continuous or semi-continuous monitoring systems, devices and methods may sample the ISF. Some embodiments of the continuous or semi-continuous monitoring systems, devices and methods may incorporate a dilution technique. Some embodiments of the continuous or semi-continuous monitoring systems, devices and methods may sample the blood. In additional aspects, the disclosure is directed to a kit including one or more of a continuous or semi-continuous analyte monitoring system, an infusion device as described herein, and instructions for using the same.

Certain embodiments are described further in the following description, examples, claims and drawings. These features of embodiments will become more apparent from the following detailed description when taken in conjunction with the accompanying exemplary drawings.

### BRIEF DESCRIPTION OF THE DRAWINGS

FIG. 1 illustrates an embodiment of a device for continuous or semi-continuous monitoring of one or more bodily analytes.

FIG. 2 illustrates an embodiment of an analyte monitoring system and method that may utilize an analyte monitoring device such as, e.g., that shown in FIG. 1.

FIG. 3 illustrates another embodiment of an analyte monitoring system and method that may utilize an analyte monitoring device such as, e.g., that shown in FIG. 1.

FIG. 4 illustrates another embodiment of an analyte monitoring system and method that may utilize an analyte monitoring device such as, e.g., that shown in FIG. 5.

FIG. 5 illustrates another embodiment of a device for continuous or semi-continuous monitoring of one or more bodily analytes.

FIG. 6 shows a graphical result of a model estimating the results of an analyte monitoring system that utilizes a dilution/microperfusion method.

The drawings illustrate embodiments of the technology and are not limiting. For clarity and ease of illustration, the drawings may not be made to scale and, in some instances, various aspects may be shown exaggerated or enlarged to facilitate an understanding of particular embodiments.

### DETAILED DESCRIPTION

Provided herein are continuous or semi-continuous analyte monitoring systems, devices and techniques that may be used alone or in conjunction with one or more remote or integrated devices, such as an infusion pump, that overcome limitations of present systems, devices and methods. Although the embodiments are described herein in the context of the monitoring and sampling of glucose and the delivery of insulin, other analytes may be monitored and sampled using the systems, devices and methods described herein and materials other than insulin may be delivered in connection therewith.

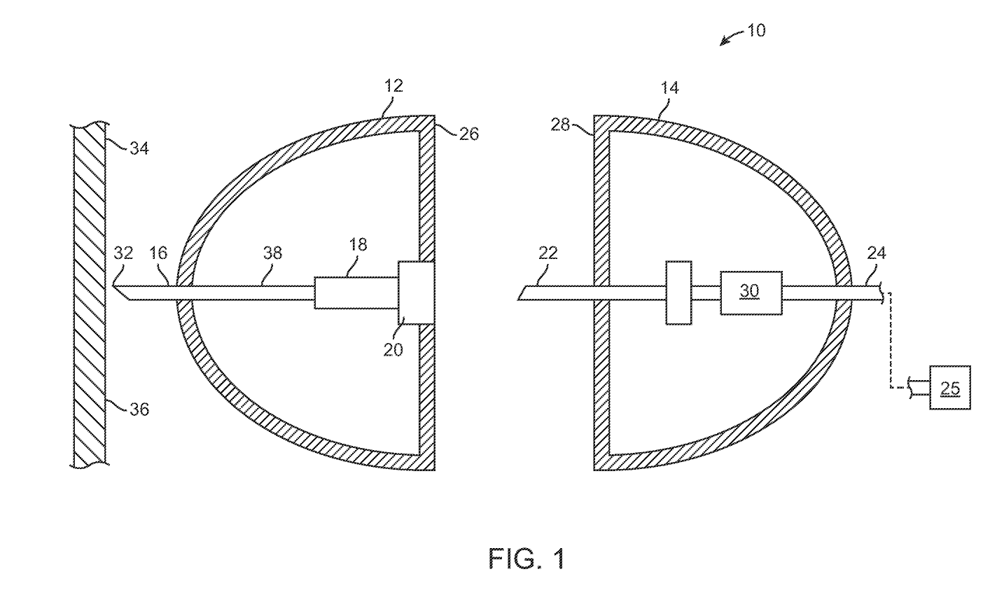

An embodiment of a device for continuous or semi-continuous monitoring of one or more bodily analytes of a patient such as, e.g., glucose, is shown in schematic cross-section in FIG. 1. The device 10 of FIG. 1 includes a proximal housing 12 and a distal housing 14 shown in cutaway view to illustrate the components therein. The proximal housing 12 contains a cannula 16 extending therefrom that is connected to a lumen 18 having a septum 20 for receiving a needle 22 that extends into the distal housing 14. The needle 22 has an inner lumen which is in fluid connection with an inner lumen of a fluid line 24 that can be in fluid communication with a pump 25, such as those described in PCT Patent Application No. PCT/US2010/043789. During use, the proximal and distal housings 12 and 14 may be detachably or releasably connected to each other via known means, such as clips, snap-fit tabs, etc., at the proximal housing interface 26 and distal housing interface 28. In FIG. 1, the proximal housing 12 and distal housing 14 are shown in a detached configuration as may be experienced when the patient is bathing, swimming, or otherwise desires to disconnect the sensor 30 (and infusion device if connected to the fluid line) from the rest of the device 10.

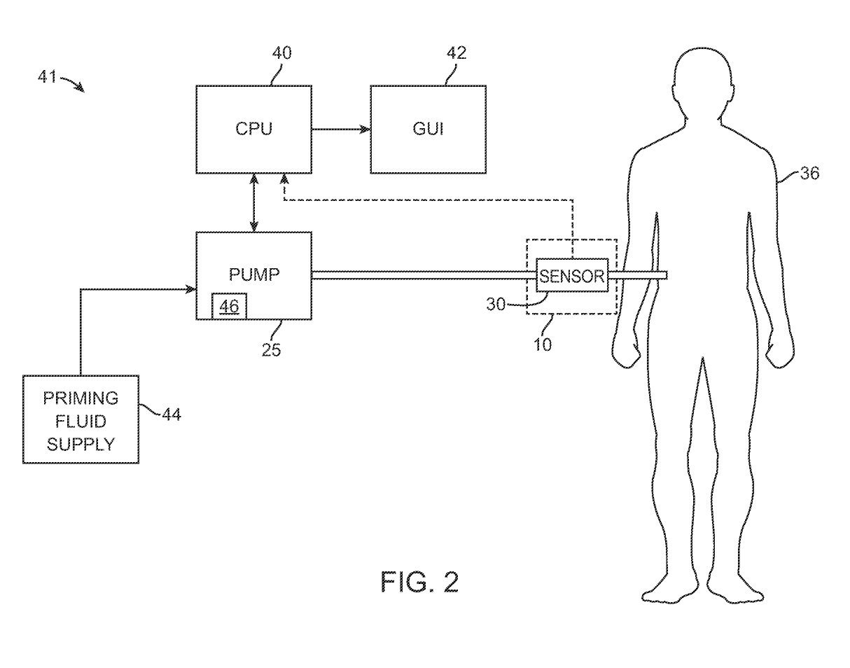

The cannula 16 as shown in FIG. 1 may be a tubular member of high strength material having a sharpened tip 32 configured for insertion through a patient's skin 34 into the patient's body 36 for inserting and withdrawing fluid into and from the patient's subcutaneous space (e.g., interstitial space), in the intramuscular space, in the dermal layers, or blood vessel. The proximal housing 12 may be affixed to the patient's body 34 in a convenient location such as the underside of the upper arm, abdomen, etc. via an adhesive component or other means (not shown) that is connected to or an integral part of the proximal housing 12 as known in the art. Within the proximal housing 12, the cannula proximal end 38 is fluidly attached to a lumen having septum 20 for receiving the needle 22 that is housed in the distal housing 14. The proximal end of the needle 22 is in fluid connection with fluid line 24 that incorporates within the distal housing 14 sensor 30 such as a glucose sensor (e.g., a GOx-based electrode) in the fluid path. This sensor 30 may be connected via a wired or wireless communications link to a processor 40 for processing data obtained by the sensor 30 to determine the concentration of analyte measured, such as glucose, and for further processing to display concentration data to a patient or caregiver, and alternatively for use with an infusion device for delivering, e.g., insulin to the patient based in part on the amount of glucose sensed. The processor 40, shown in the system embodiment 41 of FIG. 2 as a central processing unit (CPU), and graphic user interface (GUI) 42 each or both may be remotely located from the device 10 of FIG. 1; alternatively each or both may be integrated into the device 10 of FIG. 1 and, e.g., incorporated into the housing 14 of the device 10 of FIG. 1. The processor 40 and GUI 42 also each or both may be part of one or more pumps 25 such as an infusion pump.

In use, a patient or caregiver inserts the cannula 16 in the embodiment of FIG. 1 into the patient's subcutaneous space through the skin 34 using conventional techniques so that an inner lumen of the cannula 16 is in fluid communication with the patient's interstitial fluid (ISF) at the cannula distal end 32. Alternatively, the cannula 16 may be inserted into the dermal layers, intramuscular space, or a blood vessel if sampling of analyte therefrom is desired. The sharpened distal end of the tubular needle 22 is inserted through the septum 20 of the lumen 18 that is in fluid communication with the cannula 16, forming a continuous fluid column running from the patient's body 36 to the sensor 30 and, e.g., a fluid pump. Unlike previous devices, the sensor 30 is not located in the patient's body 36 but is rather in the fluid column outside the patient's body 36 in the distal housing 14 that is detachable from the proximal housing 12. In this manner, the sensor 30 may easily be replaced and is less susceptible to challenges associated with sensors based in the hostile environment of the patient's body 36.

### ISF Sampling System and Method

An embodiment of an analyte monitoring system and method is shown in FIG. 2 that can utilize an analyte monitoring device 10 such as that shown in FIG. 1 in connection with an infusion device 25, show in FIG. 2 as a “pump”, such as that described in PCT Patent Application No. PCT/US2010/043789. In this embodiment, the fluid line 24 of the monitoring device 10 of FIG. 1 is connected directly to the pump 25 and the cannula 16 of the monitoring device 10 is inserted into the interstitial space of a patient's body 36 beneath the patient's skin 34 for sampling the patient's ISF.

The pump 25 is connected to at least one priming fluid supply 42, which variously may be a known concentration of glucose in saline and/or an insulin solution for delivery to the patient 36. One or both of the pump 25 and/or sensor 30 are in communication (via., e.g., a wired, wireless, or optical connection) with a processor 40 shown in FIG. 2 as a CPU. The CPU 40 may contain a processor and computer-readable medium containing instructions when executed to process information presented by the sensor 30 regarding analyte concentration levels as well as other information as may be desired, such as, pH, concentration levels of one or more additional analytes, temperature, etc., and present such information to a processor 46 within the pump 25 and/or a separate display device operatively coupled to the CPU 40 for reading by the patient, a caregiver, or remote display device such as, e.g., a GUI 42 shown in FIG. 2. When used to monitor glucose levels of a patient 36 in connection with an infusion device such as an insulin pump 25, a closed-loop or semi-closed loop insulin delivery device based on continuous or semi-continuous glucose data of the patient 36 is possible.

In one exemplary method of use of the system depicted in FIG. 2 in which the distal end 32 of the cannula 16 is located in the subcutaneous space of the body 36 of a patient, the pump 25 is used to first prime the fluid column as described above with fluid from the priming fluid supply 44. Intermittently or continuously thereafter, the pump 25 withdraws an amount of ISF from the patient's subcutaneous space through the fluid column at or just past the sensor 30 located in the fluid path of the fluid line 24 in a quantity and location sufficient for the sensor 30 to measure the analyte of interest (e.g., ISF glucose concentration). If a pump 25 is not used, any means to withdraw the ISF for sampling by the sensor 30 may be used.

This ISF can be continuously drawn from the body 36 for continuous or semi-continuous analyte measurements (e.g., glucose concentration) at intervals desired by the patient or caregiver. This sampled ISF is then returned to the body 36 through the same fluid column by the pump 25 operating in reverse fashion when it is desired only to sample the ISF to determine the patient's glucose level, providing a system that could provide measurements at intervals and the ISF would not need to be collected as waste. The data collected by the sensor 30 may be transmitted by the communication link to the CPU 40 and to the pump 25 and/or the GUI 42. The sampled ISF may alternatively or in part be delivered to a waste collection container or drain (not shown) for disposal.

Alternatively, the embodiment of FIG. 2 may be used in connection with a medicament delivery system. For example, the analyte data obtained by the system embodiment 41 of FIG. 2 may be fed via a communications link to an insulin delivery device. In turn, such data may be used in a closed-loop or semi-closed loop fashion to allow a processor to calculate and control the delivery of medicament such as insulin in real-time or periodically based on the analyte (in this case, glucose) level sensed.

In such an arrangement where insulin delivery is desired and glucose concentration is monitored, a single cannula 16 may be used for both insulin delivery and glucose monitoring. For example, if the pump 25 in FIG. 2 is an infusion device such as that described in described in PCT Patent Application No. PCT/US2010/043789, medicament such as insulin may be used as the fluid to prime the fluid column as may be used, e.g., in connection with the delivery of a bolus of insulin solution. After the passage of some time to allow the diffusion of the insulin solution into the patient's body 36 via the cannula 16, ISF may be then withdrawn from the patient's body 36 via the cannula 16 and presented to the sensor 30 through the same fluid column by the pump 25 operating in reverse fashion, allowing the system 41 to measure the concentration of analyte, e.g., glucose, as the sampled ISF is presented to the sensor 30. All or a portion of this sampled ISF may then be returned to the body 36 through the same fluid column by the pump 25 or discarded as waste. Such a cycle of periodic ISF sampling at desired intervals accompanied by delivery of medicament in desired intervals, such as a bolus or basal levels of insulin solution, may be accomplished using the methods and systems described herein.

In another example, in an embodiment where insulin solution is provided to a patient as a basal rate with delivery every, e.g., 5 minutes, between 2 and 3 minutes after the time of such insulin delivery, a sample volume of ISF may be collected via the same fluid column of system 41 and presented to the sensor 30. The sensor 30 determines the glucose concentration in the sampled ISF and at the next insulin delivery time (e.g., 5 minutes from the previous insulin delivery time), the sampled ISF volume plus the desired incremental insulin solution volume is pushed to the patient's body 36. For example, if the required sample volume is 10 μL and the desired insulin basal delivery rate is 1.2 units of insulin (U) per hour, (12 μL/h with U100 insulin), then between 2 and 3 minutes after insulin delivery, 10 μL of ISF would be withdrawn and measured, and then at 5 min 11 μL (the 10 μL sample volume plus the 1 μL basal insulin requirement) of such fluid would be returned to the body 36.

Other examples of embodiments where an initial bolus of insulin solution is delivered followed by cycles of periodic ISF sampling and returning such sampled ISF to the patient's body 36 in connection with desired intervals of insulin solution delivery as a basal rate, with or without additional deliveries of one or more boluses of such insulin solution, may be accomplished in any combination and variety of medicament delivery and ISF sampling intervals desired.

Calibration of the sensor 30 could be augmented using any insulin delivery of significant volume. For example, the glucose concentration in the insulin solution is a known value (nearly zero). As such insulin solution may be moved to and/or past the sensor 30, the CPU 40 may use the glucose concentration obtained by the sensor 30 to estimate the calibration coefficients to align with the known concentration in the insulin. Other calibration techniques as known in the art may also or in conjunction with this technique be utilized.

In the case where the ISF is returned to the patient's body 36, an additional pumping mechanism would not be required; rather, for implementation utilizing the infusion mechanisms or pumps such as those described in PCT Patent Application No. PCT/US2010/043789, the insulin pumping mechanism can be leveraged utilizing an additional outlet port that is plumbed back to the pump's reservoir to permit reverse flow from the patient 36. Alternatively, the ‘bucket’ of such a pump's valve could be sized to provide the required reversal volume. For implementations with conventional syringe pump drive mechanisms, a plunger of such a syringe-type pump can retract to provide the reversed flow.

This back and forth action of the liquid described in this method can also provide substantial benefits in reducing the time to detect occlusions at low medication delivery rates. By pulling and pushing this sample volume at each measurement interval, the time to detect an occlusion should be no longer than a measurement interval, as opposed to the systems that purely deliver the medication at low rates that may require hours to detect an occlusion.

### Diffusion Method

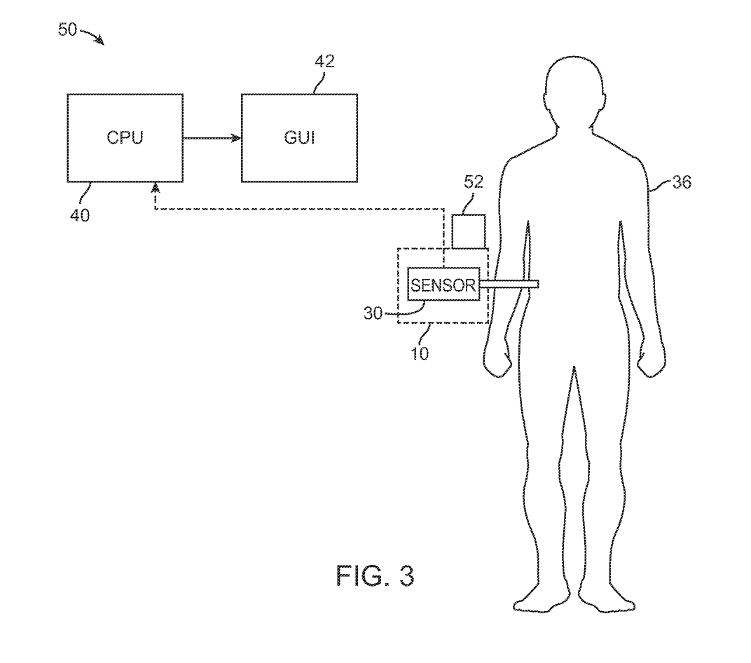

Another embodiment of an analyte monitoring system 50 and method is shown in FIG. 3; this embodiment can also utilize an analyte monitoring device 10 such as that shown in FIG. 1 and in its simplest configuration includes an analyte monitoring device 10 in communication with a processor such as a CPU 40 and a display device such as a GUI 42. The processor 40 and GUI 42 each or both may be remotely located from the device 10 of FIG. 1; alternatively each or both 40 and 42 may be integrated into the device 10 of FIG. 1 and, e.g., incorporated into the housing 14 of the device 10 of FIG. 1. The processor 40 and GUI 42 also each or both may be part of one or more pumps such as an infusion pump 25. During use, as described above in connection with the embodiment 41 of FIG. 2, the cannula 16 may be inserted into the patient's body 36 at a desired location so that its distal end 32 is in the patient's interstitial space or other desired location of the body. Rather than using a pump 25 or other means to create a flow of a priming fluid supply through the cannula 16 to bring the ISF sample up to the sensor 30, however, the system 50 of this embodiment relies on diffusion of the ISF through the fluid column to the sensor 30 sufficient to allow analyte measurements to be made by the sensor 30 and to be sent to the CPU 40 via a communications link as described above. The sensor 30 may be located closer to the cannula 16 or in the cannula fluid path (but not such that it is indwelling within the patient's body 36) so to facilitate the diffusion method by not requiring the ISF to diffuse as far into the fluid column as when the sensor 30 is located in the fluid line 24 as shown in FIG. 1.

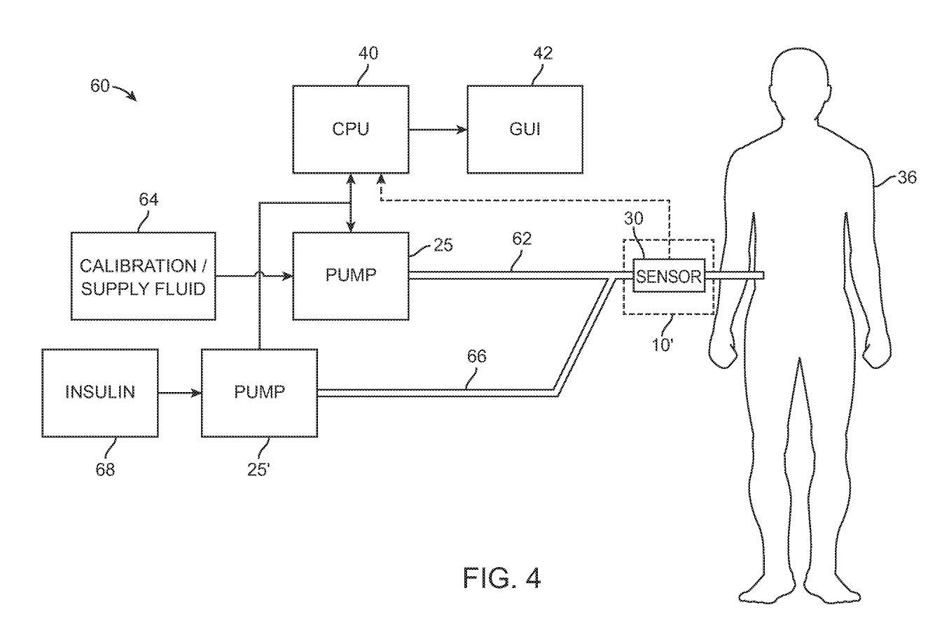

Alternatively, a pump 25 or like mechanism may be attached to fluid line 24 (such as that shown in the embodiment of FIG. 2 or 4) to assist with the diffusion method. The ISF diffusing into the fluid column via the cannula 16 could be perturbated by mechanically pulsing a drive mechanism of a pump 25 attached to the fluid line 24. In addition, a secondary mechanism could be incorporated to provide independent action. The secondary mechanism 52 could be a vibratory motor, piezo elements, or other mechanisms well known in the art. This can have the effect of accelerating the diffusion of the analyte through the fluid column to the sensor 30 by adding a secondary convective transport mechanism. Such perturbations could additionally be used to allow monitoring of occlusions as previously described.

### Dilution/Microperfusion Method

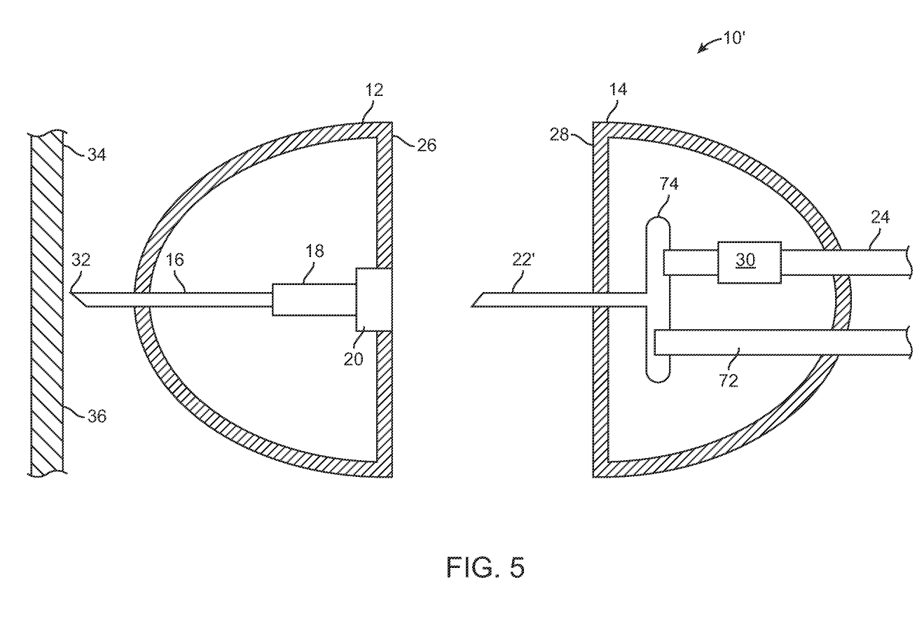

Another embodiment of an analyte monitoring system 60 and method is shown in FIG. 4; this embodiment 60 may utilize an analyte monitoring device such as that shown in FIG. 5. A first pump 25 is shown in fluid communication with the analyte monitoring device 10 via a first fluid line 62 as described below, and one or both of the first pump and/or sensor 30 are in communication (via., e.g., a wired, wireless, or optical connection) with a CPU 40 and a display device such as a GUI 42. The processor 40 and GUI 42 each or both may be remotely located from the device 10 of FIG. 5; alternatively each or both 40 and 42 may be integrated into the device 10 of FIG. 5 and, e.g., incorporated into the housing 14 of the device 10 of FIG. 5. The processor 40 and GUI 42 also each or both may be part of one or more pumps 25 such as an infusion pump. This first pump 25 is in fluid connection with a source of calibration fluid 64 and need not be an infusion pump for the delivery of medicament. The embodiment 60 shown in FIG. 4 optionally is integrated with a medicament pump 25¹ (such as described, e.g., in PCT Patent Application No. PCT/US2010/043789) that is in fluid communication with the analyte monitoring device 10 via a second fluid line 66 (as described below in connection with the embodiment 60 of FIG. 5). The medicament pump 25¹ may also be in fluid connection with a source of medicament 68 such as a solution of insulin and may be in communication via a communication link with the CPU 40 as shown in FIG. 4.

FIG. 5 shows another embodiment of the analyte monitoring device 10¹ that may be used in connection with the systems and methods described herein and in particular with the system 60 and method described in connection with FIG. 4. This analyte monitoring device 10¹ includes many of the features common to the device 10 described in connection with FIG. 1. The device 10¹ of FIG. 5 includes a second fluid line 72 at least partially disposed within the distal housing 14 along with a first fluid line 24. Each of the first and second fluid lines 24 and 72 has distal ends in fluid communication with a mixing chamber 74 that in turn is in fluid communication with the proximal end of a needle 22¹. When the needle 22¹ is disposed within the septum 20 as previously described, a fluid column is created that allows the movement of fluid such as ISF to and from the body 36 via the first pump 25 and/or second pump 25¹.

In the embodiment of FIG. 4, a quantity of calibration/supply fluid, which may be, e.g., a known concentration of glucose in saline, may be used by the first pump 25 optionally to prime the first fluid line 62 of the analyte monitoring device 60. Intermittently at desired intervals, a “dilution volume” of this fluid may be delivered by first pump 25 via first fluid line 62 into the patient at the site of the body 36 in which the cannula 16 has been inserted and is allowed to equilibrate for a time with the surrounding ISF. During this equilibration step, in the example of glucose monitoring, glucose present in the patient's ISF will diffuse into this fluid (some of the fluid will be absorbed by the body 36).

Next, a “sample volume” of the dilution volume (which may be all or a portion of the dilution volume or even an amount greater than the dilution volume as supplemented by available ISF) may be pulled through the analyte monitoring device 10¹ by the first pump 25 through the fluid column 62 to a point that is at or beyond the sensor 30 so to allow the sensor 30 to measure the analyte (e.g., glucose) concentration. The analyte concentration should be representative of that present in the ISF. This process is repeated to provide analyte (e.g., glucose) measurements intermittently in intervals as desired by the patient or caregiver. If the entire dilution volume is withdrawn to provide the measurement sample volume, then little or no net delivery of supply fluid is provided to the patient. If only a portion of the dilution volume is withdrawn, additional fluid is required to continue to provide this dilution volume into the patient's body 36.

This dilution technique can also be implemented utilizing the same cannula 16 for both insulin delivery and glucose monitoring. In the embodiment 10¹ of FIG. 5, the insulin delivery can be made by the medicament pump 25¹ via the second fluid line 66, the sample volume is withdrawn to the sensor 30 in the first fluid line by the first pump 25 and would reverse direction for the delivery of the dilution volume.

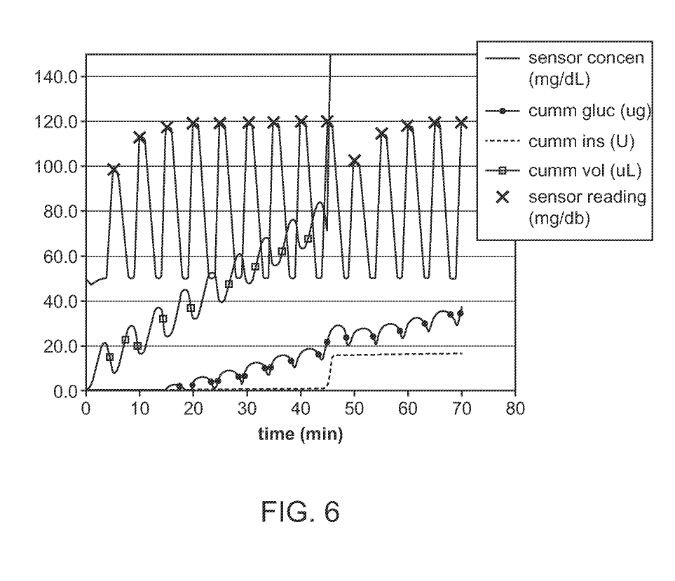

The graphical results of a mathematical model shown in FIG. 6 plots an illustrative estimate of fluid volume as a function of time. Here, the system 60 begins with fresh solution and a portion of that solution is reused multiple times as the dilution volume. In this illustrative estimation, approximately 20 μL is infused into a patient's subcutaneous space and 14 μL is withdrawn by the system 60 for sampling. Also, a 2 unit per hour basal rate (2 U/h) is included and a 15 unit bolus is delivered. Calibration of the sensor 30 may be assisted by the addition of the solution. The glucose concentration of this solution can be a known concentration. In the estimate above, it is assumed that the concentration is 50 mg/dl. On each delivery of the dilution volume, the sensor 30 may be presented with fresh solution and the calibration of the sensor 30 may be ensured. The concentration of the provided calibration/supply fluid may be selected at the low alarm limit. In this manner, the accuracy of the low alarm (arguably the most important detection value for glucose monitoring) would be easily detected with high confidence. In this embodiment, the sensor would respond with a direction change as the measured value crosses across the alarm value.

Infusion of insulin solution or other medicament for this embodiment can be accomplished utilizing a ‘double’ spool pump using a single pump mechanism (one outlet pushing insulin for one lumen, and another port for the other lumen pushing and pulling the supply solution. Alternatively, an independent pumping mechanism could be utilized to push and pull from the supply solution, independent of the insulin pumping mechanism. This back and forth pumping action may provide similar improvements in occlusion detection performance as previously described.

While particular forms of embodiments have been illustrated and described, it will be apparent that various modifications can be made without departing from the spirit and scope of the embodiments herein. Accordingly, it is not intended that the invention be limited by the forgoing detailed description.

The entirety of each patent, patent application, publication and document referenced herein is hereby incorporated by reference. Citation of the above patents, patent applications, publications and documents is not an admission that any of the foregoing is pertinent prior art, nor does it constitute any admission as to the contents or date of these documents.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” may refer to one of or a plurality of the elements it modifies (e.g., “a reagent” can mean one or more reagents) unless it is contextually clear either one of the elements or more than one of the elements is described. Although the present technology has been specifically disclosed by representative embodiments and optional features, modification and variation of the concepts herein disclosed may be made, and such modifications and variations may be considered within the scope of this technology.

What is claimed is:

1. An analyte monitoring system, comprising:
a proximal housing configured to be affixed to a patient's body;
a cannula that is configured for placement through the skin of a patient's body, that extends from the proximal housing, and that includes an inner lumen; and
one or more continuous or semi-continuous sensors placed in fluid communication with the inner lumen of the cannula such that the one or more sensors are not in direct contact with the body of the user.

2. The system of claim 1 wherein the one or more sensors comprise at least one glucose sensor.

3. The system of claim 1 wherein the cannula is configured to be placed through the skin of a patient's body into any one of the subcutaneous space, the intramuscular space, the dermal layers, or a venous blood vessel.

4. The system of claim 2 wherein the sensor comprises glucose oxidase (GOx) based electrodes or fluorescence based devices.

5. The system of claim 1 further comprising a distal housing that may be detachably connected to the proximal housing.

6. The system of claim 5 wherein the proximal housing comprises a septum in fluid communication with the cannula and distal housing comprises a needle configured to penetrate the septum of the proximal housing upon engagement of the housings.

7. The system of claim 1 further comprising a pump in fluid communication with the inner lumen of the cannula and configured to move fluid out of the patient's body, through the inner lumen of the cannula and into fluid communication with the one or more sensors.

8. The system of claim 1 further comprising a processor and user interface configured for processing measurements taken by the one or more sensors and transmitting the processed measurements to a user interface to present the measurements to a patient or caregiver.

9. The system of claim 8 wherein the processor is configured to transmit the processed measurements to a recording medium.

10. The system of claim 1 further comprising a processor configured for processing measurements taken by the one or more sensors and transmitting the processed measurements via a communications link to a remote device.

11. The system of claim 10 wherein the communications link comprises a wireless link, an optical link or a wired link.

12. The system of claim 10 wherein the remote device comprises a user interface for presenting the measurements to a patient or caregiver.

13. The system of claim 10 wherein the remote device comprises an infusion pump.

14. The system of claim 13 wherein the remote infusion device comprises an insulin pump.

15. The system of claim 1 further comprising a processor configured for processing measurements taken by the one or more sensors and transmitting the processed measurements to an integrated infusion device.

16. The system of claim 13 wherein the integrated infusion device comprises an insulin pump.

17. The system of claim 1 wherein the cannula is configured to sample ISF.

18. The system of claim 1 wherein the cannula is configured to transport fluid from the patient's body into fluid communication with the one or more sensors by diffusion.

19. The system of claim 18 further comprising a secondary mechanism configured to facilitate movement interstitial fluid out of the patient's body into cannula and into fluid communication with the one or more sensors by diffusion.

20. A kit comprising:
one or more of a continuous or semi-continuous analyte monitoring system of claim 1;
an infusion device; and
instructions for using the same.

21. An analyte monitoring system, comprising:
a cannula which has an inner lumen and which is configured to be placed through the skin into the subcutaneous space of the patient's body;
a proximal housing that may be affixed to a patient's body which comprises a septum that is in fluid communication with an inner lumen of the cannula and that is disposed at a distal end of the cannula;
a distal housing that may be detachably secured to the proximal housing and which comprises a needle configured to penetrate the septum of the proximal housing upon engagement of the housings; and
one or more continuous or semi-continuous sensors placed in fluid communication with the inner lumen of the cannula such that the one or more sensors are not in direct contact with the patient's body.

22. The system of claim 21 further comprising a pump in fluid communication with the inner lumen of the cannula configured to move interstitial fluid out of the patient's body into cannula and into fluid communication with the one or more sensors.

23. The system of claim 21 further comprising a secondary mechanism configured to facilitate movement interstitial fluid out of the patient's body into cannula and into fluid communication with the one or more sensors by diffusion.

24. The system of claim 21 wherein the one or more sensors comprise any one of glucose oxidase (GOx) based electrodes or fluorescence based sensors.

25. The system of claim 21 wherein the cannula is configured to sample ISF of the patient's body.
